(19) United States
(12) Patent Application Publication
(10) Pub. No.: US 2012/0087845 A1
(43) Pub. Date: Apr. 12, 2012

(54) STEAM STERILIZATION SYSTEM FOR A WATER PURIFIER

(76) Inventors: Yong Jin Kim, Gyeonggi-do (KR); Han Sik Kim, Jangsu-gun Jeollabuk-do (KR)

(21) Appl. No.: 13/376,668

(22) PCT Filed: Jun. 7, 2010
(86) PCT No.: PCT/KR10/03619

§ 371 Date: Dec. 7, 2011

(30) Foreign Application Priority Data
Jun. 8, 2009 (KR) 10-2009-0050655
Jun. 7, 2010 (KR) 10-2010-0053128

(51) Int. Cl. A61L 2/07

(52) U.S. Cl. 422/292

(57) ABSTRACT

The present invention relates to a sterilization system for a water purifier, i.e., a system which can sterilize, using hot water and steam, the interiors of a filter, a water tank, a cooling tank, a hot water tank, lines connected to each unit, and a water nozzle of the water purifier, which are vulnerable to bacteria. The system of the present invention effectively sterilizes the water purifier to enable users to use a highly clean water purifier.

### TECHNICAL FIELD

The present invention relates to a sterilization system for a water purifier. The water purifier may be contaminated by bacteria in the course of purification of water as time goes past. Accordingly, the water purifier should be sterilized and cleaned on a regular basis, which is very complicated and requires technique that makes ordinary people almost unable to do by themselves. As a result, professional after service personnel replace filters and sterilize and clean the water purifier, but such work does not sterilize the water purifier perfectly.

### BACKGROUND ART

Due to such disadvantages, the filter means of water purifiers are contaminated by bacteria and ordinary users use such water purifiers whose hygiene is at a risky level, as broadcast by the media. Whenever the bacteria of water purifiers are broadcast on TV, there arises a hygiene problem, but such problem is easily forgotten as time goes past.

### DISCLOSURE

### Technical Problem

The present invention has been made to solve the problems and it is an object of the present invention to provide a means which effectively sterilizes various filter means, a water tank, a cooling tank and a hot water tank within a water purifier to keep the water purifier clean.

### Technical Solution

In order to achieve the object of the present invention, a steam sterilization system for a water purifier has a steam generator provided in a line where purifier water flows and enables steam to flow in a filter line, a water tank, a cooling tank and a hot water tank for sterilization.

### ADVANTAGEOUS EFFECT

As described above, a steam sterilization system for a water purifier according to the present invention enables steam to flow in a filter line, a water tank, a cooling tank, a hot water tank and a water nozzle along a water purification line for an effective sterilization, and removes impurities from the filter line as well as sterilizing the water purifier by performing a steam sterilization operation on a regular basis under management of a user.

### BRIEF DESCRIPTION OF DRAWINGS

FIG. 1 illustrates a steam sterilization system for a water purifier which sterilizes the water purifier by providing steam or hot water to each unit of the water purifier by using a steam device means 55 of the steam sterilization system for the water purifier according to the present invention.

FIG. 2 illustrates the steam sterilization system for the water purifier which sterilizes the water purifier by providing hot water of a hot water tank means 76 to each unit of the water purifier in the steam sterilization system for the water purifier according to the present invention.

### DESCRIPTION OF REFERENCE NUMERALS FOR MAJOR PARTS OF DRAWINGS

Original water entrance (5) Sol valve means (6)

Triple fitting connection means (7) Wastewater sol valve means (8)

Filter means (9) Sol valve means (10)

Quadruple fitting connection means (11) Steam sol valve means (12)

Steam triple fitting connection means (13) Wastewater sol valve means (14)

Wastewater triple fitting connection means (15) Sol valve means (16)

Input pump means (17) Sol valve means (18)

Quadruple fitting connection means (19) Steam sol valve means (20)

Steam triple fitting connection means (21) Wastewater sol valve means (22)

Wastewater triple fitting connection means (23) Sole valve means (24)

Filter means (25) Sol valve means (26)

Quadruple fitting connection means (27) Steam sol valve means (28)

Steam triple fitting connection means (29) Wastewater sol valve means (30)

Wastewater triple fitting connection means (31) Sol valve means (32)

Filter means (33)

Check valve means (35) Pressure switch means (36)

Sol valve means (37) Quadruple fitting connection means (38)

Steam sol valve means (39) Steam triple fitting connection means (40)

Wastewater sol valve means (41) Wastewater triple fitting connection means (42)

Sol valve means (43) Filter means (44)

Auto shutoff means (45) Steam sol valve means (46)

Wastewater triple fitting connection means (47) Wastewater resistance means (48)

Wastewater discharger (49) Sol valve means (50)

Triple fitting connection means (51) Sol valve means (52)

Triple fitting connection means (53) Sol valve means (54)

Steam device means (55) Triple fitting connection means (56)

Sol valve means (57) water tank means (58)

Internal space unit (59) Inlet (60)

Vertical ball top means (61) Outlet (62)

Triple fitting connection means (63) Sol valve means (64)

Cold water tank means (65) Internal space unit (66)

Inlet (67) Vertical ball top means (68)

Outlet (69) Sol valve means (70)

Water nozzle button switch (71) Water nozzle lever switch (72)

Water nozzle (73) Outlet (74)

Sol valve means (75) Hot water tank means (76)

Internal space unit (77) Inlet (78)

Vertical ball top means (79) Outlet (80)

Sol valve means (81) Water nozzle button switch (82)

Water nozzle lever switch (83) Water nozzle (84)

Outlet (85) Sol valve means (86)

Triple fitting connection means (87) Triple fitting connection means (88)

Pump means (89) Wastewater sol valve means (90)

Water purifier hose means (100)

### BEST MODE

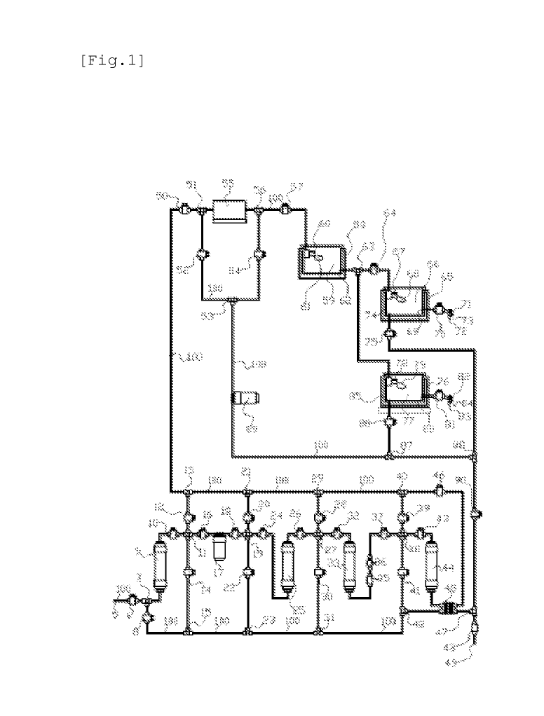

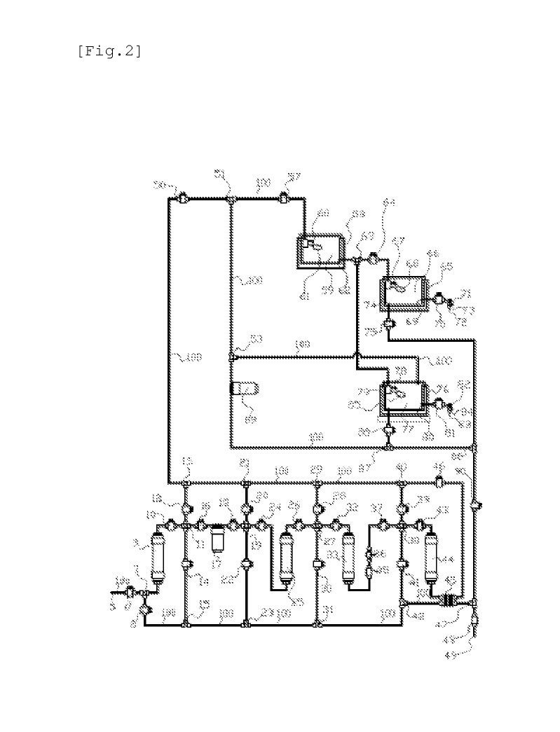

Hereinafter, configuration and exemplary embodiments of the present invention will be described in detail with accompanying drawings. In FIG. 1, water which is entered through an original water entrance 5 becomes clean after passing through a filter means 9, a filter means 25, a filter means 33, and a filter means 44, and then stored in a water tank means 58, a cold water tank means 65 and a hot water tank means 76. Whenever necessary, the stored water is discharged through a water nozzle 73 and a water nozzle 84 for use. A water purifier according to the present invention further includes a plurality of sol valve means which is provided between each filter and each unit for control. All of parts of the water purifier according to the present invention are connected by a water purifier hose. The water purifier hose which connects the parts will be collectively referred to as a water purifier hose means 100. Each of the parts of the water purifier according to the present invention is connected by the water purifier hose means 100, in which water flows and steam passes. Accordingly, even if the water purifier hose means 100 is not mentioned in explaining each part of the water purifier according to the present invention, those parts are connected by the water purifier hose means 100. The original water entrance 5 is connected to a sol valve means 6, which is connected to a triple fitting connection means 7 to be divided into two parts. The original water is connected to the filter means 9 and to a wastewater sol valve means 8 to flow in a wastewater line direction. The wastewater is discharged through a wastewater triple fitting connection means 15, a wastewater triple fitting connection means 23, a wastewater triple fitting connection means 31, a wastewater triple fitting connection means 42, a wastewater triple fitting connection means 47, and a wastewater resistance means 48 to a wastewater discharger 49 which are configured for a wastewater line. The water which is purified by the filter means 9 may flow in any of four directions through the sol valve means 10 as connectors are provided in four directions in the quadruple fitting connection means 11. The water may be connected to the wastewater discharger 49 through the wastewater triple fitting connection means 14, the wastewater sol valve means 15 in the wastewater line, or connected to the steam device means 55 as intended by being connected to a steam connector of the steam triple fitting connection means 13, a steam triple fitting connection means 21, a steam triple fitting connection means 29, and a steam triple fitting connection means 40 through the steam sol valve means 12. If the water is connected to the sol valve means 16 from the quadruple fitting connection means 11, it is connected to the sol valve means 18 and the quadruple fitting connection means 19 through an input pump means 17. As connectors are provided in four directions in the quadruple fitting connection means 19, the water may be connected to the wastewater triple fitting connection means 23 and the wastewater discharger 49 through the wastewater sol valve means 22. If the water is connected to the steam sol valve means 20 through the quadruple fitting connection means 19, it is connected to the steam device means 55 through the steam triple fitting connection means 21. If the water is connected to the sol valve means 24 through the quadruple fitting connection means 19, it is connected to the quadruple fitting connection means 27 through the filter means 25 and the sol valve means 26. As water may be connected to four directions from the quadruple fitting connection means 27, it may be connected to the wastewater discharger 49 through the wastewater sol valve means 30 and the wastewater triple fitting connection means 31. If the water is connected to the steam sol valve means 28 from the quadruple fitting connection means 27, it is connected to the steam device means 55 through the steam triple fitting connection means 29. If the water flows to the sol valve means 32 from the quadruple fitting connection means 27, it is connected to the filter means 33. Then, the water may be connected to the quadruple fitting connection means 38 through a check valve means 35 and a pressure switch means 36, which may be provided as necessary, and the sol valve means 37. As connectors are provided in four directions in the quadruple fitting connection means 38, the water may be connected to the wastewater discharger 49 through the wastewater sol valve means 41 and a wastewater triple fitting connection means 42 from the quadruple fitting connection means 38. The water may be connected to the steam device means 55 from the quadruple fitting connection means 38 through the steam sol valve means 39 and the steam triple fitting connection means 40. The water may be connected to the filter means 44 through the sol valve means 43 from the quadruple fitting connection means 38. The water is connected to the steam device means 55 through an auto shutoff means 45 and the steam sol valve means 46 as necessary. Then, the water may be connected to the steam device means 55 through a steam triple fitting connection means 40, a steam triple fitting connection means 29, a steam triple fitting connection means 21 and a steam triple fitting connection means 13, a sol valve means 50 and a triple fitting connection means 51. If a small quantity of current flows to the steam device means 55 through an electric heating means, the water flowing in the steam device means 55 becomes hot promptly. If a large quantity of current flows to the steam device means 51 to provide a large quantity of heat, the water flowing in the steam device means 55 becomes steam promptly. The steam device means 55 may be heated by applying current to a heat wire, which is also called a resistance wire, and by an electric induction method by applying a high frequency to an outside of the steam device means 55. The steam device means 55 is connected a water tank means 58 through the triple fitting connection means 56 and the sol valve means 57. The steam device means 55 is then connected to an inlet 60 of an internal space unit 59 of the water tank means 58. The flow of water may be controlled by a vertical ball top means 61. The water may be connected to a cold water tank means 65 or to a hot water tank means 76 through the outlet 62 of the internal space unit 59 and the triple fitting connection means 63. The water may be connected to the cold water tank means 65 through the sol valve means 64. The cold water tank means 65 controls the water which is supplied through the inlet 67 of the internal space unit 66 by the vertical ball top means 68. The water is controlled by the sol valve means 70 through the outlet 69 and then purified cold water is discharged through the water nozzle 73 for use. Operating the water nozzle button switch 71 and the water nozzle lever switch 72 may control the sol valve means 70. The sol valve means 70 may be open or closed by a control of the water purifier. The water is connected in a direction of the steam device means 55 through the outlet 74 of the internal space unit 66, the sol valve means 75 and the triple fitting connection means 88 and to the wastewater discharger 49 through the wastewater sol valve means 90. The water is connected to the hot water tank means 76 from the triple fitting connection means 63 and then connected to the inlet 78 of the internal space unit 77 to be controlled by the vertical ball top means 79. The water is controlled by the sol valve means 81 through the outlet 80 and then hot water may be discharged through the water nozzle 84 for use. The hot water may be discharged by opening the sol valve means 81 by the operation of the water nozzle button switch 82 and the water nozzle lever switch 83. The sol valve means 81 may be open or closed by a control of the water purifier. The water is connected to the triple fitting connection means 87 through the outlet 85 of the internal space unit 77 and the sol valve means 86. Then, the water may be connected to the wastewater discharger 49 from the triple fitting connection means 87 or to the steam device means 55 in any direction through the pump means 89, the triple fitting connection means 53, the sol valve means 52 and the sol valve means 54. The water stored in the water tank means 58, the cold water tank means 65 and the hot water tank means 76 may be directly discharged to the wastewater discharger 49 or to be supplied to the steam device means 55 to become steam. The aforementioned filter means 9, filter means 25, filter means 33, and filter means 44 may employ all of filter means used in the water purifier. The filter means 9, 25, 33, and 44 may include a sediment pre-filter, a pre-carbon filter, an ultra filtration filter, a post carbon filter, a CSM filter, a reverse osmotic pressure filter, a sediment filter, a TCR filter, a CSM U/F filter or an R/O filter, but not limited thereto. While a water purifying operation is performed for almost 24 hours, a steam sterilization operation is performed for only a couple of minutes or dozens of minutes in operating the steam sterilization system for the water purifier according to the present invention. Accordingly, the water purification operation accounts for most of operation hours. As the steam sterilization system for the water purifier according to the present invention employs a number of sol valve means, power consumption is great if power is supplied to coils of all of the sol valve means. This may give burden to homes and business places. To reduce power consumption of the sol valve means, application of the sol valve means should be consistent with purpose of use. The sol valve means is divided into those closed in a normal state and those open in a normal state. That is, the sol valve means is classified into a sol valve means which are open and closed in a normal state and expressed as no and nc and do not operate as power is not supplied to the coil. Due the reason described above, when the steam sterilization system for the water purifier according to the present invention performs the water purification operation, the following sol valve means may employ nc sol valve means: the wastewater sol valve means 8, the wastewater sol valve means 14, the wastewater sol valve means 22, the wastewater sol valve means 30, the wastewater sol valve means 41, the steam sol valve means 12, the steam sol valve means 20, the steam sol valve means 28, the steam sol valve means 39, the sol valve means 52, the sol valve means 54, the sol valve means 86, the sol valve means 75 and the wastewater sol valve means 90 which are closed in a normal state to purify water introduced by the original water entrance 5. The following sol valve means may employ no sol valve means: the sol valve means 6, the sol valve means 10, sol valve means 16, the sol valve means 18, the sol valve means 24, the sol valve means 26, the sol valve means 32, the sol valve means 37, the sol valve means 43, the steam sol valve means 46, the sol valve means 50, the sol valve means 57, and the sol valve means 64 which should be open always during the water purification operation. Then, the water which is entered by the original water entrance 5 is purified through the sol valve means 6, the triple fitting connection means 7, the filter means 9, the sol valve means 10, the quadruple fitting connection means 11, the sol valve means 16, the input pump means 17, the sol valve means 18, the quadruple fitting connection means 19, the sol valve means 24, the filter means 25, the sol valve means 26, the quadruple fitting connection means 27, the sol valve means 32, the filter means 33, the check valve means 35, the pressure switch means 36, the sol valve means 37, the quadruple fitting connection means 38, the sol valve means 43 and the filter means 44. Then, the purified water is collected in the water tank means 58 and then in the cold water tank means 65 and the hot water tank means 76 through the auto shutoff means 45, the steam sol valve means 46, the steam triple fitting connection means 40, the steam triple fitting connection means 29, the steam triple fitting connection means 21, the steam triple fitting connection means 13, the sol valve means 50, the triple fitting connection means 51, the steam device means 55, the triple fitting connection means 56, and the sol valve means 57 to be used as cold water and hot water as intended. When the water purification operation is performed, power is not supplied to the steam device means 55 not to generate steam. If it is the time for a steam sterilization operation as programmed by a user while the water purification operation is performed by the foregoing method, the steam sterilization operation is performed as follows. The sol valve means 6 is driven and closed to shut off original water, and the sol valve means 10, the sol valve means 18, the sol valve means 26 and the sol valve means 37 are driven and closed, and the wastewater sol valve means 41 is driven and open to be connected in the direction of the wastewater discharger 49. The sol valve means 57 is driven and closed to shut off the steam from being supplied to the water tank means 58. The steam device means 55 is driven and heated and the sol valve means 75, the sol valve means 86 and the sol valve means 54 are driven and open to connect water from the internal space unit 59 of the water tank means 58, from the internal space unit 66 of the cold water tank means 65 and from the internal space unit 77 of the hot water tank means 76 to the steam device means 55. If the pump means 89 is driven, the water from the water tank means 58, the cold water tank means 65 and the hot water tank means 76 is supplied to the steam device means 55 in a direction opposite to the direction for the water purification operation and becomes steam. The steam flows to and sterilizes the triple fitting connection means 51, the sol valve means 50, the steam triple fitting connection means 13, the steam triple fitting connection means 21, the steam triple fitting connection means 29, the steam triple fitting connection means 40, the steam sol valve means 46, the auto shutoff means 45 and the filter means 44 in a reverse direction. In particular, the steam pushes impurities from the filter means 44 in a reverse direction as well as sterilizing the filter means 44. Thus, the steam flows to and sterilizes the sol valve means 43, the quadruple fitting connection means 38, the wastewater sol valve means 41, and the wastewater triple fitting connection means 42 and pushes impurities toward the wastewater discharger 49. If the sterilization and cleaning of the filter means 44 is finished, the wastewater sol valve means 41, the sol valve means 43 and the steam sol valve means 46 are closed, and the wastewater sol valve means 30, the sol valve means 32, the sol valve means 37, and the steam sol valve means 39 are open. Then, the steam flows from the steam triple fitting connection means 40 to, and sterilizes, the steam sol valve means 39, the quadruple fitting connection means 38, the sol valve means 37, the pressure switch means 36, the check valve means 35, the filter means 33, the sol valve means 32, the quadruple fitting connection means 27, the wastewater sol valve means 30, the wastewater triple fitting connection means 31 and the wastewater discharger 49. In particular, the steam flows to the filter means 33 in a reverse direction and pushes impurities from the filter means 33 to the wastewater discharger 49 as well as sterilizing the filter means 33 to thereby sterilize and clean up the filter means 33. If the cleaning of the filter means 33 is finished, the wastewater sol valve means 30, the sol valve means 32, the and the steam sol valve means 39 are closed, and the wastewater sol valve means 22, the sol valve means 24, the sol valve means 26 and the steam sol valve means 28 are open. Then, the steam flows from the steam triple fitting connection means 29 to, and sterilizes the steam sol valve means 28, the quadruple fitting connection means 27, the sol valve means 26, the filter means 25, the sol valve means 24, the quadruple fitting connection means 19, the wastewater sol valve means 22, the wastewater triple fitting connection means 23 and the wastewater discharger 49. In particular, the steam simultaneously sterilizes and pushes impurities from the filter means 25 while flowing in the filter means 25 to thereby sterilize and clean up the filter means 25. If the cleaning of the filter means 25 is finished, the wastewater sol valve means 22, the sol valve means 24, and the steam sol valve means 28 are closed and the wastewater sol valve means 14, the sol valve means 16, the sol valve means 18 and the steam sol valve means 20 are open to receive the steam from the steam triple fitting connection means 21. Then, the steam flows to and sterilizes the steam sol valve means 20, the quadruple fitting connection means 19, the sol valve means 18, the input pump means 17, the sol valve means 16, the quadruple fitting connection means 11, the wastewater sol valve means 14, the wastewater triple fitting connection means 15 and the wastewater discharger 49 in a reverse direction. In particular, the steam sterilizes the input pump means 17 and the line thereof. If the cleaning is finished, the wastewater sol valve means 14, the sol valve means 16 and the steam sol valve means 20 are closed, and the wastewater sol valve means 8, the sol valve means 10 and the steam sol valve means 12 are open. Then, the steam flows to and sterilizes and cleans the steam triple fitting connection means 13, the steam sol valve means 12, the quadruple fitting connection means 11, the sol valve means 10, the filter means 9, the triple fitting connection means 7, the wastewater sol valve means 8, the wastewater triple fitting connection means 15 and the wastewater discharger 49. In particular, the steam sterilizes the filter means 9 and removes impurities from the filter means 9. That is, the steam sterilization system for the water purifier according to the present invention completely sterilizes the environment which is easily contaminated by bacteria and impurities such as the filter means 9, 25, 33 and 44 and pushes the impurities in a reverse direction. The steam sterilization system for the water purifier according to the present invention may sterilize and remove impurities from units which frequently contact purified water and are contaminated by bacteria such as the input pump means 17 as necessary. If the sterilization and cleaning operation for each filter line is finished, the sol valve means 50 and the sol valve means 54 are closed and the sol valve means 52 and the sol valve means 57 are open to shut off the steam from flowing in the direction of the filter line and to flow from the steam device means 55 to the triple fitting connection means 56, the sol valve means 57 and the water tank means 58. As the sterilization effect is maximized when there is no water in the internal space unit 59, the water should be removed from the water tank means 58 and cold water tank means 65 while a small quantity of water is remained in the hot water tank means 76 prior to the sterilization operation if water is consumed or not consumed for the cleaning of the filter line. To remove water therefrom, the sol valve means 64, the sol valve means 75 and the wastewater sol valve means 90 are open to discharge water to the wastewater discharger 49 to empty the internal space units 59 and 66. Then, the internal space units 59 and 66 are sterilized by steam. In this case, the sol valve means 70 is open so that the steam is discharged to and sterilizes the water nozzle 73. After the steam sterilization and cleaning is performed by the foregoing method, the water purification operation may be performed as necessary. Otherwise, the temperature of the steam device means 55 is decreased and the sterilization and cleaning operation may be performed by using hot water instead of steam by the foregoing method. Otherwise, the hot water may be used first for the sterilization and cleaning operation. The hot water tank means 76 makes and stores therein hot water and is not easily contaminated by bacteria. However, as necessary, the hot water tank means 76 may be sterilized and cleaned by opening the sol valve means 81 and the sol valve means 86, removing the remaining water therefrom, discharging steam through the water nozzle 84 and the triple fitting connection means 87. If the sterilization and cleaning operation is finished, the sol valve means may be open or closed to perform the water purification operation as described in exemplary embodiments as above. The aforementioned water purification, sterilization and cleaning operations are automatically performed by a control of a control box, and a user may simply input the sterilization and cleaning time or press a button at necessary time. FIG. 2 illustrates the same configuration as in FIG. 1 except that the hot water of the hot water tank means 76 is used for sterilization and cleaning operations, instead of the steam device means 55, or the steam is generated by increasing the temperature of the hot water tank means 76 to be used for the sterilization and cleaning operations. The usage method in FIG. 2 is the same or partly similar to that in FIG. 1, and description thereof will be omitted. Also, instead of the steam device means 55, the hot water tank means 76 may be additionally provided and installed to configure the steam sterilization system for the water purifier. Among the aforementioned filter means, filters which are shut off in a reverse direction may be sterilized by steam supplied in a forward direction. The steam device means 55 may be installed in the original water entrance 5 to supply steam in a forward direction and sterilize each unit according to the water purification line as necessary. A pressure sensor (not shown) and/or a check valve (not shown) may be provided in both sides of the steam device means 55 to detect a pressure of steam that is higher than a proper level and control the electric system of the steam device means 55, or provided between each of the filter means.

1. A steam sterilization system for a water purifier which comprises an original water entrance, a sol valve means, a triple fitting connection means, a wastewater sol valve means, a filter means, a sol valve means, a quadruple fitting connection means, a steam sol valve means, a steam triple fitting connection means, a wastewater sol valve means, a wastewater triple fitting connection means, a sol valve means, an input pump means, a sol valve means, a quadruple fitting connection means, a steam sol valve means, a steam triple fitting connection means, a wastewater sol valve means, a wastewater triple fitting connection means, a sol valve means, a filter means, a sol valve means, a quadruple fitting connection means, a steam sol valve means, a steam triple fitting connection means, a wastewater sol valve means, a wastewater triple fitting connection means, a sol valve means, a filter means, a check valve means, a pressure switch means, a sol valve means, a quadruple fitting connection means, a steam sol valve means, a steam triple fitting connection means, a wastewater sol valve means, a wastewater triple fitting connection means, a sol valve means, a filter means, an auto shutoff means, a steam sol valve means, a wastewater triple fitting connection means, a wastewater resistance means, a wastewater discharger, a sol valve means, a triple fitting connection means, a sol valve means, a triple fitting connection means, a sol valve means, a steam device means, a triple fitting connection means, a sol valve means, a water tank means, an internal space unit, an inlet, a vertical ball top means, an outlet, a triple fitting connection means, a sol valve means, a cold water tank means, an internal space unit, an inlet, a vertical ball top means, an outlet, a sol valve means, a water nozzle button switch, a water nozzle lever switch, a water nozzle, an outlet, a sol valve means, a hot water tank means, an internal space unit, an inlet, a vertical ball top means, an outlet, a sol valve means, a water nozzle button switch, a water nozzle lever switch, a water nozzle, an outlet, a sol valve means, a triple fitting connection means, a triple fitting connection means, a pump means, a wastewater sol valve means and a water purifier hose means and a plurality of sol valve means between filters and between units for control, and has the water purifier hose means connected between parts to flow water and steam therein, wherein the original water entrance is connected to the sol valve means, and the sol valve means is connected to the tripe fitting connection means which is divided into two directions so that original water is connected to the filter means and the wastewater sol valve means to flow to the wastewater discharger through the wastewater fitting connection means, the wastewater triple fitting connection means, the wastewater triple fitting connection means, the wastewater triple fitting connection means, the wastewater triple fitting connection means, the wastewater resistance means which are provided in a wastewater line, and the water purified by the filter means is connected to any of four directions of the quadruple fitting connection means through the sol valve means, and connected to the wastewater discharger through the wastewater sol valve means and the wastewater triple fitting connection means of the wastewater line or connected to a steam connector of the steam triple fitting connection means through the steam sol valve means, the steam triple fitting connection means, the steam triple fitting connection means, and the steam triple fitting connection means to be connected to the steam device means as intended, or connected to the quadruple fitting connection means through the quadruple fitting connection means, the sol valve means, the input pump means and the sol valve means, and then connected to the wastewater discharger through the wastewater sol valve means, and the wastewater triple fitting connection means as connectors are provided in four directions in the quadruple fitting connection means, or connected from the quadruple fitting connection means to the steam device means through the steam sol valve means, and the steam triple fitting connection means, or connected from the quadruple fitting connection means to the quadruple fitting connection means through the sol valve means, the filter means, and the sol valve means, and then connected from the quadruple fitting connection means to the wastewater discharger through the wastewater sol valve means and the wastewater triple fitting connection means, or connected from the quadruple fitting connection means to the steam device means through the steam sol valve means and the steam triple fitting connection means, or connected from the quadruple fitting connection means to the filter means through the sol valve means or connected to the quadruple fitting connection means through the check valve means, the pressure switch means and the sol valve means, and then connected from the quadruple fitting connection means to the wastewater discharger through the wastewater sol valve means and the wastewater triple fitting connection means as connectors are provided in four directions in the quadruple fitting connection means, or connected from the quadruple fitting connection means to the steam device means through the steam sol valve means and the steam triple fitting connection means and connected to the filter means from the quadruple fitting connection means through the sol valve means, or connected to the steam device through the auto shutoff means and the steam sol valve means as necessary, and connected to the steam device means from the steam triple fitting connection means, the steam triple fitting connection means, the steam triple fitting connection means, and the steam triple fitting connection means through the sol valve means and the triple fitting connection means, wherein the steam device means heats water promptly if a small quantity of current is supplied by an electric heating means and converts the water into steam promptly if a large quantity of current is supplied with a large quantity of heat to thereby sterilize the water purifier hose means, the filter means, the filter means, the filter means and the filter means by steam.

2. A steam sterilization system for a water purifier which comprises an original water entrance, a sol valve means, a triple fitting connection means, a wastewater sol valve means, a filter means, a sol valve means, a quadruple fitting connection means, a steam sol valve means, a steam triple fitting connection means, a wastewater sol valve means, a wastewater triple fitting connection means, a sol valve means, an input pump means, a sol valve means, a quadruple fitting connection means, a steam sol valve means, a steam triple fitting connection means, a wastewater sol valve means, a wastewater triple fitting connection means, a sol valve means, a filter means, a sol valve means, a quadruple fitting connection means, a steam sol valve means, a steam triple fitting connection means, a wastewater sol valve means, a wastewater triple fitting connection means, a sol valve means, a filter means, a check valve means, a pressure switch means, a sol valve means, a quadruple fitting connection means, a steam sol valve means, a steam triple fitting connection means, a wastewater sol valve means, a wastewater triple fitting connection means, a sol valve means, a filter means, an auto shutoff means, a steam sol valve means, a wastewater triple fitting connection means, a wastewater resistance means, a wastewater discharger, a sol valve means, a triple fitting connection means, a sol valve means, a triple fitting connection means, a sol valve means, a steam device means, a triple fitting connection means, a sol valve means, a water tank means, an internal space unit, an inlet, a vertical ball top means, an outlet, a triple fitting connection means, a sol valve means, a cold water tank means, an internal space unit, an inlet, a vertical ball top means, an outlet, a sol valve means, a water nozzle button switch, a water nozzle lever switch, a water nozzle, an outlet, a sol valve means, a hot water tank means, an internal space unit, an inlet, a vertical ball top means, an outlet, a sol valve means, a water nozzle button switch, a water nozzle lever switch, a water nozzle, an outlet, a sol valve means, a triple fitting connection means, a triple fitting connection means, a pump means, a wastewater sol valve means and a water purifier hose means, wherein the steam device means is connected to the water tank means through the triple fitting connection means and sol valve means, and connected to the inlet provided in the internal space unit of the water tank means and a flow of water is controlled by the vertical ball top means and then the steam device means is connected to the cold water tank means and the hot water tank means through the outlet of the internal space unit and the triple fitting connection means and connected to the cold water tank means through the sol valve means, and the water which is introduced to the inlet of the internal space unit is controlled by the vertical ball top means and then controlled by the sol valve means through the outlet so that the purified cold water is discharged through the water nozzle for use, and a control means is provided to open and close the sol valve means by the operation of the water nozzle button switch and water nozzle lever switch and by a control of the water purifier, and the water is connected in the direction of the steam device means through the outlet of the internal space unit, the sol valve means and the triple fitting connection means and to the wastewater discharger through the wastewater sol valve means, and connected from the triple fitting connection means to the inlet of the internal space unit through the hot water tank means to be controlled by the vertical ball top means and controlled by the sol valve means through the outlet, and then used as hot water by being discharged through the water nozzle, and the hot water is discharged by opening the sol valve means by the operation of the water nozzle button switch and the water nozzle lever switch, and the sol valve means is open and closed by a control of the water purifier, and the water is connected to the wastewater discharger through the outlet of the internal space unit, the sol valve means, the triple fitting connection means or connected to the steam device means in any direction through the pump means, the triple fitting connection means, the sol valve means, and the sol valve means, and the water which is stored in the water tank means, the cold water tank means and the hot water tank means is directly discharged to the wastewater discharger and supplied to the steam device means to become steam.

3. A steam sterilization system for a water purifier which comprises an original water entrance, a sol valve means, a triple fitting connection means, a wastewater sol valve means, a filter means, a sol valve means, a quadruple fitting connection means, a steam sol valve means, a steam triple fitting connection means, a wastewater sol valve means, a wastewater triple fitting connection means, a sol valve means, an input pump means, a sol valve means, a quadruple fitting connection means, a steam sol valve means, a steam triple fitting connection means, a wastewater sol valve means, a wastewater triple fitting connection means, a sol valve means, a filter means, a sol valve means, a quadruple fitting connection means, a steam sol valve means, a steam triple fitting connection means, a wastewater sol valve means, a wastewater triple fitting connection means, a sol valve means, a filter means, a check valve means, a pressure switch means, a sol valve means, a quadruple fitting connection means, a steam sol valve means, a steam triple fitting connection means, a wastewater sol valve means, a wastewater triple fitting connection means, a sol valve means, a filter means, an auto shutoff means, a steam sol valve means, a wastewater triple fitting connection means, a wastewater resistance means, a wastewater discharger, a sol valve means, a triple fitting connection means, a sol valve means, a triple fitting connection means, a sol valve means, a steam device means, a triple fitting connection means, a sol valve means, a water tank means, an internal space unit, an inlet, a vertical ball top means, an outlet, a triple fitting connection means, a sol valve means, a cold water tank means, an internal space unit, an inlet, a vertical ball top means, an outlet, a sol valve means, a water nozzle button switch, a water nozzle lever switch, a water nozzle, an outlet, a sol valve means, a hot water tank means, an internal space unit, an inlet, a vertical ball top means, an outlet, a sol valve means, a water nozzle button switch, a water nozzle lever switch, a water nozzle, an outlet, a sol valve means, a triple fitting connection means, a triple fitting connection means, a pump means, a wastewater sol valve means and a water purifier hose means, wherein the sol valve means is driven and closed to shut off original water, and the sol valve means, the sol valve means, the sol valve means and the sol valve means are driven and closed and the wastewater sol valve means is driven and open to be connected in the direction of the wastewater discharger, and the sol valve means is driven and closed to shut off steam from flowing in the direction of the water tank means, and the steam device means is driven and heated and the sol valve means, the sol valve means and the sol valve means are driven and open to connect water from the internal space unit of the water tank means, from internal space unit of the cold water tank means and from the internal space unit of the hot water tank means to the steam device means, and the pump means is driven to supply water from the water tank means, the cold water tank means and the hot water tank means to the steam device means in a direction opposite to that for the water purification operation to generate steam, and the steam flows to and sterilizes the triple fitting connection means, the sol valve means, the steam triple fitting connection means, the steam triple fitting connection means, the steam triple fitting connection means, the steam triple fitting connection means, the steam sol valve means, the auto shutoff means and the filter means in a reverse direction, and in particular, sterilizes and pushes impurities from the filter means in a reverse direction, and sterilizes the sol valve means, the quadruple fitting connection means, the wastewater sol valve means, and the wastewater triple fitting connection means and pushes impurities to the wastewater discharger, and after the completion of the sterilization and cleaning of the filter means, and the wastewater sol valve means, the sol valve means and the steam sol valve means are closed and the wastewater sol valve means, the sol valve means, the sol valve means and the steam sol valve means are open to enable the steam to flow from the steam triple fitting connection means to and sterilize the steam sol valve means, the quadruple fitting connection means, the sol valve means, the pressure switch means, the check valve means, the filter means, the sol valve means, the quadruple fitting connection means, the wastewater sol valve means, the wastewater triple fitting connection means and the wastewater discharger, and in particular, the steam flows to and sterilizes the filter means in a reverse direction and pushes impurities therefrom to the wastewater discharger for sterilization and cleanup of the filter means, and after the completion of the cleanup of the filter means, the wastewater sol valve means, the sol valve means, and the steam sol valve means are closed and the wastewater sol valve means, the sol valve means, the sol valve means and the steam sol valve means are open to enable the steam to flow from the steam triple fitting connection means to the steam sol valve means, the quadruple fitting connection means, the sol valve means, the filter means, the sol valve means, the quadruple fitting connection means, the wastewater sol valve means, the wastewater triple fitting connection means and the wastewater discharger and in particular sterilize and pushes impurities from the filter means in a reverse direction for sterilization and cleanup of the filter means, and after the completion of the cleanup of the filter means, the wastewater sol valve means, the sol valve means and the steam sol valve means are closed and the wastewater sol valve means, the sol valve means, the sol valve means and the steam sol valve means are open to enable steam to flow from the steam triple fitting connection means to the steam sol valve means, the quadruple fitting connection means, the sol valve means, the input pump means, the sol valve means, the quadruple fitting connection means, the wastewater sol valve means, the wastewater triple fitting connection means and the wastewater discharger for sterilization in a reverse direction and sterilize the input pump means and the line thereof, and after the completion of the cleanup, and the wastewater sol valve means, the sol valve means, and the steam sol valve means are closed and the wastewater sol valve means, the sol valve means and the steam sol valve means are open to flow steam from the steam triple fitting connection means to the steam sol valve means, the quadruple fitting connection means, the sol valve means, the filter means, the triple fitting connection means, the wastewater sol valve means, the wastewater triple fitting connection means and the wastewater discharger in a reverse direction for sterilization and cleanup at the same time.

4. A steam sterilization system for a water purifier which comprises an original water entrance, a sol valve means, a triple fitting connection means, a wastewater sol valve means, a filter means, a sol valve means, a quadruple fitting connection means, a steam sol valve means, a steam triple fitting connection means, a wastewater sol valve means, a wastewater triple fitting connection means, a sol valve means, an input pump means, a sol valve means, a quadruple fitting connection means, a steam sol valve means, a steam triple fitting connection means, a wastewater sol valve means, a wastewater triple fitting connection means, a sol valve means, a filter means, a sol valve means, a quadruple fitting connection means, a steam sol valve means, a steam triple fitting connection means, a wastewater sol valve means, a wastewater triple fitting connection means, a sol valve means, a filter means, a check valve means, a pressure switch means, a sol valve means, a quadruple fitting connection means, a steam sol valve means, a steam triple fitting connection means, a wastewater sol valve means, a wastewater triple fitting connection means, a sol valve means, a filter means, an auto shutoff means, a steam sol valve means, a wastewater triple fitting connection means, a wastewater resistance means, a wastewater discharger, a sol valve means, a triple fitting connection means, a sol valve means, a triple fitting connection means, a sol valve means, a steam device means, a triple fitting connection means, a sol valve means, a water tank means, an internal space unit, an inlet, a vertical ball top means, an outlet, a triple fitting connection means, a sol valve means, a cold water tank means, an internal space unit, an inlet, a vertical ball top means, an outlet, a sol valve means, a water nozzle button switch, a water nozzle lever switch, a water nozzle, an outlet, a sol valve means, a hot water tank means, an internal space unit, an inlet, a vertical ball top means, an outlet, a sol valve means, a water nozzle button switch, a water nozzle lever switch, a water nozzle, an outlet, a sol valve means, a triple fitting connection means, a triple fitting connection means, a pump means, a wastewater sol valve means and a water purifier hose means, wherein the sol valve means and sol valve means are closed and the sol valve means and the sol valve means are open to shut off steam from flowing in the direction of the filter line and to flow steam from the steam device means to triple fitting connection means, the sol valve means and the water tank means, and the water is discharged to the wastewater discharger from the water tank means and cold water tank means by opening the sol valve means, the sol valve means and the wastewater sol valve means while a small quantity of water is remained in the hot water tank means to thereby empty the internal space unit and the internal space unit for steam sterilization and the sol valve means is open to discharge steam to the water nozzle.

5. The steam sterilization system for the water purifier according to claim 1, wherein the steam device means is heated by applying a current to a heat wire, which is also called a resistance wire and heated by applying a high frequency by an electric induction method from the outside of the steam device means in heating the steam device means.

6. The steam sterilization system for the water purifier according to claim 1, wherein the temperature of the steam device means is decreased to make hot water instead of steam to perform a cleanup operation by the method stated in claim 1 and the cleanup operation is performed by using hot water instead of steam from the first time.

7. The steam sterilization system for the water purifier according to claim 1, wherein the sterilization and cleanup operations are performed by using hot water of the hot water tank means instead of the steam device means or performed by using steam by increasing the temperature of the hot water tank means or the hot water tank means is additionally provided and a sol valve means and a control device means are provided.

8. The steam sterilization system for the water purifier according to claim 2, wherein the steam device means is heated by applying a current to a heat wire, which is also called a resistance wire and heated by applying a high frequency by an electric induction method from the outside of the steam device means in heating the steam device means.

9. The steam sterilization system for the water purifier according to claim 2, wherein the temperature of the steam device means is decreased to make hot water instead of steam to perform a cleanup operation by the method stated in claim 2 and the cleanup operation is performed by using hot water instead of steam from the first time.

10. The steam sterilization system for the water purifier according to claim 2, wherein the sterilization and cleanup operations are performed by using hot water of the hot water tank means instead of the steam device means or performed by using steam by increasing the temperature of the hot water tank means or the hot water tank means is additionally provided and a sol valve means and a control device means are provided.

11. The steam sterilization system for the water purifier according to claim 3, wherein the steam device means is heated by applying a current to a heat wire, which is also called a resistance wire and heated by applying a high frequency by an electric induction method from the outside of the steam device means in heating the steam device means.

12. The steam sterilization system for the water purifier according to claim 3, wherein the temperature of the steam device means is decreased to make hot water instead of steam to perform a cleanup operation by the method stated in claim 3 and the cleanup operation is performed by using hot water instead of steam from the first time.

13. The steam sterilization system for the water purifier according to claim 3, wherein the sterilization and cleanup operations are performed by using hot water of the hot water tank means instead of the steam device means or performed by using steam by increasing the temperature of the hot water tank means or the hot water tank means is additionally provided and a sol valve means and a control device means are provided.

14. The steam sterilization system for the water purifier according to claim 4, wherein the steam device means is heated by applying a current to a heat wire, which is also called a resistance wire and heated by applying a high frequency by an electric induction method from the outside of the steam device means in heating the steam device means.

15. The steam sterilization system for the water purifier according to claim 4, wherein the temperature of the steam device means is decreased to make hot water instead of steam to perform a cleanup operation by the method stated in claim 4 and the cleanup operation is performed by using hot water instead of steam from the first time.

16. The steam sterilization system for the water purifier according to claim 4, wherein the sterilization and cleanup operations arc performed by using hot water of the hot water tank means instead of the steam device means or performed by using steam by increasing the temperature of the hot water tank means or the hot water tank means is additionally provided and a sol valve means and a control device means are provided.
